SEQUENCE LISTING (5 sequences)

SEQ ID NO: 1 - DNA, 6153 bp, Artificial Sequence
  Shuttle vector that replicates in Rhodococcus and E.coli
cacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttggaattctccgcgcaccaccgacacatttcgaccgaaggacatcgatgcttccggaccgactgaccgcgctcgcctccgccgcacacaacgccggagcacgtgagaacctccacaacctcggcggcgtccaccacgacgcacaccacgccaccctcaccctcgatcgctacacacccgagaagctcccagcgtcctcctgggccgcgatactcgaccaccacgcacgcacaccgcactaacgattcggccggcgctcgattcggccggcgctcgattcggccggcgctcgattcggccggcgctcgattcggccggcgctcgattcggccgagcagaagagtgaacaaccaccgaccacgcttccgctctgcgcgccgtacccgacctacctcccgcagctcgaagcagctcccgggagtaccgccgtactcacccgcctgtgctcaccatccaccgacgcaaagcccaacccgagcacacctcttgcaccaaggtgccgaccgtggctttccgctcgcagggttccagaagaaatcgaacgatccagcgcggcaaggttcaaaaagcaggggttggtggggaggaggttttggggggtgtcgccgggatacctgatatggctttgttttgcgtagtcgaataattttccatatagcctcggcgcgtcggactcgaatagttgatgtgggcgggcacagttgccccatgaaatccgcaacggggggcgtgctgagcgatcggcaatgggcggatgcggtgttgcttccgcaccggccgttcgcgacgaacaacctccaacgaggtcagtaccggatgagccgcgacgacgcattggcaatgcggtacgtcgagcattcaccgcacgcgttgctcggatccatcgtcatcgactgcgatcacgtcgacgccgcgatgcgcgcattcgagcaaccatccgaccatccggcgccgaactgggtcgcacaatcgccgtccggccgcgcacacatcggatggtggctcggccccaaccacgtgtgccgcaccgacagcgcccgactgacgccactgcgctacgcccaccgcatcgaaaccggcctcaagatcagcgtcggcggcgatttcgcgtatggcgggcaactgaccaaaaacccgattcaccccgattgggagacgatctacggcccggccaccccgtacacattgcggcagctggccaccatccacacaccccggcagatgccgcgtcggcccgatcgggccgtgggcctgggccgcaacgtcaccatgttcgacgccacccggcgatgggcatacccgcagtggtggcaacaccgaaacggaaccggccgcgactgggaccatctcgtcctgcagcactgccacgccgtcaacaccgagttcacgacaccactgccgttcaccgaagtacgcgccaccgcgcaatccatctccaaatggatctggcgcaatttcaccgaagaacagtaccgagcccgacaagcgcatctcggtcaaaaaggcggcaaggcaacgacactcgccaaacaagaagccgtccgaaacaatgcaagaaagtacgacgaacatacgatgcgagaggcgattatctgatgggcggagccaaaaatccggtgcgccgaaagatgacggcagcagcagcagccgaaaaattcggtgcctccactcgcacaatccaacgcttgtttgctgagccgcgtgacgattacctcggccgtgcgaaagctcgccgtgacaaagctgtcgagctgcggaagcaggggttgaagtaccgggaaatcgccgaagcgatggaactctcgaccgggatcgtcggccgattactgcacgacgcccgcaggcacggcgagatttcagcggaagatctgtcggcgtaaccaagtcagcgggttgtcgggttccggccggcgctcggcactcggaccggccggcggatggtgttctgcctctggcgcagcgtcagctaccgccgaaggcctgtcatcgaccggcttcgactgaagtatgagcaacgtcacagcctgtgattggatgatccgctcacgctcgaccgctacctgttcagctgccgcccgctgggcatgagcaacggccaactctcgttcaagatccgcgatccgctggacatggtcaaaatcagggcggccatgaccgtgggcaccttcgggcgggctgggctgtctcttatacacatctcaaccatcatcgatgaattgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 2 - DNA, 24 bp, Artificial Sequence
  primer
gacacatttcgaccgaaggacatc

SEQ ID NO: 3 - DNA, 26 bp, Artificial Sequence
  primer
cacggttgatgagagctttgttgtag

SEQ ID NO: 4 - DNA, 17 bp, Artificial Sequence
  primer
actggccgtcgttttac

SEQ ID NO: 5 - DNA, 19 bp, Artificial Sequence
  primer
catggtcatagctgtttcc

(19) United States
(12) Patent Application Publication
(10) Pub. No.: US 2006/0263868 A1
(43) Pub. Date: Nov. 23, 2006

(54) Rhodococcus - E. coli shuttle vector

(75) Inventors: Sanghyun Lee, Daejeon (KR); Junhyeong Cho, Daejeon (KR); Ohjin Park, Daejeon (KR); Joowon Rhee, Daejeon (KR); Si Jae Park, Daejeon (KR)

Correspondence Address: MCKENNA LONG & ALDRIDGE LLP, 1900 K STREET, NW, WASHINGTON, DC, 20006, US

(73) Assignee: LG CHEM, LTD., Seoul (KR)

(21) Appl. No.: 11/355,198

(22) Filed: Feb. 16, 2006

(30) Foreign Application Priority Data
Feb. 16, 2005 (KR) 10-2005-12892

(51) Int. Cl. C12N 1/21; C12N 15/74

(52) U.S. Cl. 435/252.2; 435/488

(57) ABSTRACT

A novel Rhodococcus-E. coli shuttle vector comprising an origin of replication of a novel plasmid derived from Rhodococcus erythropolis IAM1484 and an origin of replication derived from E. coli, is disclosed. This shuttle vector replicates bidirectionally in E. coli and Rhodococcus. Also disclosed is a recombinant vector in which a target gene is operably linked to the shuttle vector, and microorganisms transformed with the recombinant vector. The shuttle vector is relatively small in size, can replicate in a variety of species of the genus Rhodococcus and has high replication stability in a host cell. Therefore, this shuttle vector is useful for cloning a variety of genes.

### CROSS-REFERENCE TO RELATED APPLICATION

This application claims the benefit of Korean Patent Application No. 10-2005-0012892 filed on Feb. 16, 2005, in the Korean Intellectual Property Office, the disclosure of which is incorporated by reference herein in its entirety.

### BACKGROUND OF THE INVENTION

1. Field of the Invention

The present invention relates to a novel Rhodococcus-Escherichia coli (E. coli) shuttle vector comprising an origin of replication of a novel plasmid derived from Rhodococcus erythropolis IAM1484 and a DNA origin of replication derived from E. coli, which can replicate bidirectionally in E. coli and Rhodococcus, a recombinant vector in which a target gene is operably linked to the Rhodococcus-E. coli shuttle vector, and a microorganism transformed with the recombinant vector.

2. Description of the Background Art

Microorganisms of the genus Rhodococcus produce biosurfactant (Philp, J. C. et al., Appl. Microbiol. Biotechnol. 59:318, 2002) and antibiotics (Hu, T. L., Water Sci. Technol., 47:169, 2003), etc., and also biodegrade steroid compounds, bioconvert xenobiotic compounds and nitrile compounds (U.S. Pat. No. 5,135,858) and degrade acrylic acid (U.S. Pat. No. 5,998,180; U.S. patent application Ser. No. 11/224,314). Since they have a broad range of metabolic activities as above, their importance has been gradually appreciated.

In light of their importance, genetic and physiologic research of the Rhodococcus microorganisms has been widely conducted (Rahman, M. T. et al., Vet. Microbiol. 94:143, 2003). However, study of Rhodococcus metabolism and that of similar microorganisms have been very limited due to absence of appropriate host/vector systems (Finnerty, Annu. Rev. Microbiol. 46:193, 1992). Accordingly, an effective host/vector system for the genus Rhodococcus is required to produce strains having high metabolic activity.

U.S. Pat. No. 4,952,500 teaches a Rhodococcus-E. coli shuttle vector using a circular plasmid obtained from the Rhodococcus H13-A. U.S. Pat. No. 4,920,054 teaches a shuttle vector that replicates in Rhodococcus equi, Corynebacterium, Bacillus subtilis and Staphylococcus aureus, and uses a Rhodococcus origin of replication. U.S. Pat. No. 5,654,180 also teaches a shuttle vector comprising an origin of replication derived from plasmids pRC001, pRC002, pRC003 or pRC004 that can replicate in Rhodococcus rhodochrous and an origin of replication derived from plasmids pHSG299, pHSG298, pUC19 or pUC18 that can replicate in E. coli. Korean Patent Pub. KP 1999-048213 teaches a shuttle vector comprising a DNA origin of replication derived from pEk of E. coli, a DNA origin of replication derived from pCSP21197 of Rhodococcus rhodochrous, an ampicillin resistance gene as a selection marker when the vector is expressed in E. coli, and a kanamycin resistance gene as a selection marker when the vector is expressed in Rhodococcus rhodochrous. Japanese Patent Pub. JP 8-056669 teaches a shuttle vector which can be bidirectionally replicated in Rhodococcus and E. coli, and which comprises a DNA origin of replication derived from E. coli, and a DNA origin of replication which can replicate in the genus Rhodococcus derived from plasmids pNC500 or pNC 903 from a strain belonging to nocardiform bacteria.

There are additional reports of shuttle vectors using plasmids derived from Rhodococcus fascians (Desomer et al., J. Bacteriol. 170:2401, 1998; Desomer et al., Appl. Environ. Microbiol. 56:2818, 1990), Rhodococcus erythropolis (JP 10248578; EP 757101; JP 09028379; U.S. Pat. No. 5,705,386; De Mot et al., Microbiol. 146:3137, 1997), Rhodococcus rhodochrous (Kulakov et al., Plasmid 38:61, 1997), Rhodococcus equi (Zeng et al., Plasmid 38:180, 1997), and Rhodococcus sp. (PCT Publication WO 89/07151; U.S. Pat. No. 4,952,500; Vogt Singer et al., J. Bacteriol. 170:638, 1988; Appl. Environ. Microbiol. 64:4363, 1998).

Despite the development of the above shuttle vectors, they have only been used in fundamental research. So, until the present invention, there have been no commercially available tools which enable gene manipulation in Rhodococcus or microorganisms similar to Rhodococcus. This is because replicases and replication proteins that enable replication of plasmids in these particular hosts have not been known.

Although some proteins used in preparation of an expression vector or shuttle vector are known (Denis-Larose et al., Appl. Environ. Microbiol. 64:4363, 1998; Billington, et al., J. Bacteriol. 180:3233, 1998; Dasen, G. H., GI:3212128; and Mendes et al., GI:6523480), their number and utility are very limited. Although the replication stability of Rhodococcus shuttle vectors is very important, there are almost no shuttle vectors having outstanding replication stability. In many cases, to ensure replication stability of a plasmid for commercial purposes, use must be made of antibiotics which is either not economical or results in safety problems. In other words, there is no specific knowledge of the mechanism of maintaining replication stability without introducing any undesired antibiotics or related proteins.

To solve these problems, there is a need in the art for a shuttle vector that enables cloning of various genes, encodes strong replication proteins and has high replication stability in a variety of hosts.

### SUMMARY OF THE INVENTION

The present invention was intended to solve the above problems. An objective of the present invention is to provide a shuttle vector having high replication stability without requiring the use of antibiotics. This enables replication in various hosts since the vector comprises a gene sequence encoding an important protein for plasmid replication.

Another objective of the present invention is to provide (a) a recombinant vector in which a target gene is operably linked to the shuttle vector and (b) microorganisms of the genus Rhodococcus transformed with the recombinant vector.

To achieve the above objectives, the present inventors have devised a Rhodococcus-E. coli shuttle vector comprising a DNA origin of replication derived from E. coli, a DNA origin of replication derived from Rhodococcus erythropolis, an ampicillin resistance gene as a selection marker expressed in E. coli, and a kanamycin resistance gene as a selection marker expressed in Rhodococcus.

In the present invention, the DNA origin of replication from Rhodococcus erythropolis is preferably derived from the plasmid of Rhodococcus erythropolis IAM1484, and the plasmid is preferably the one designated pIAM1484.

The nucleotide sequence in one shuttle vector embodiment preferably has at least 95% sequence homology or identity with that of pJW1484 or SEQ ID NO:1. In another embodiment, the shuttle vector has at least 95% sequence homology or identity to SEQ ID NO:1 and is preferably the plasmid pLG1484.

The present invention also provides a recombinant vector in which a target gene is operably linked to the shuttle vector and the plasmid transforms microorganisms of the genus Rhodococcus.

The above and other features and embodiments of the present invention will be more fully apparent from the following detailed description and appended claims.

### BRIEF DESCRIPTION OF THE DRAWINGS

FIG. 1 is a restriction enzyme map of plasmid pIAM1484 derived from Rhodococcus erythropolis IAM1484.

FIG. 2 is a schematic drawing describing a process for preparing shuttle vector pJW1484 from plasmid pIAM1484 and pBluescript SK(−).

FIG. 3 is a schematic drawing describing a process for preparing the shuttle vector pLG1484.

FIG. 4 is a graph showing the replication stability of the shuttle vector pLG1484.

### DETAILED DESCRIPTION OF THE PREFERRED EMBODIMENT

In one aspect, the present invention relates to a novel Rhodococcus-E. coli shuttle vector that replicates efficiently in a Rhodococcus species that can be used industrially and thus serve as an important strain.

In the present invention, Rhodococcus-E. coli shuttle vector(pJW1484) was prepared using plasmid pIAM1484 isolated and purified from Rhodococcus erythropolis IAM1484 and plasmid pBluescript SK(−) widely used in E. coli. Only those regions contributing to replication and stability were finally selected, and the shuttle vector pLG1484 was prepared using them. The shuttle vector pLG1484 was then shown to replicate in various microorganisms and to have high replication stability.

To prepare the shuttle vector according to present invention, first, a cryptic plasmid was isolated from Rhodococcus erythropolis IAM1484. A search for a restriction site in the plasmid revealed the presence of an EcoRI site. So, this cryptic plasmid and E. coli plasmid pBluescript SK(−) were digested with EcoRI for cloning into E. coli. The DNA sequence of the cryptic plasmid part of the cloned plasmid was determined using a transposon, and then, an origin of replication and replicase region assumed to be important for replication were identified. Plasmid pJW1484 comprising an incidental kanamycin resistance gene was thereby obtained.

For effective transfer to a host and convenience of genetic manipulation, shuttle vector pLG1484 was prepared by deleting from the pJW1484 sequence those segments that are not involved in replication.

The inventors examined the host range of pLG1484 for many species of the genus Rhodococcus and replication stability in Rhodococcus erythropolis LG12, and determined that the shuttle vector of present invention contained strong replication proteins and had high replication stability in various hosts.

The shuttle vector according to the present invention is useful for introducing a target gene into a microorganism of the genus Rhodococcus. Therefore, in another aspect, the present invention is related to a recombinant vector in which a target gene is operably linked to the shuttle vector. Rhodococcus microorganisms are transformed with the recombinant vector.

The introduction of the target gene into the shuttle vector according to the present invention is achieved by conventional methods. To facilitate introduction of the target gene, a conventional synthetic oligonucleotide adaptor or linker can be used.

Also, to increase the expression of the target gene, expression control sequences can be added. In the present invention, “expression control sequence” means a DNA sequence that is essential for expression of an operably linked coding sequence in a certain host. This control sequence contains a promoter for transcription, an arbitrary operator sequence for the control of transcription, a sequence encoding an appropriate mRNA ribosome-binding region, and a control sequence for termination of transcription and translation.

A nucleic acid is linked operably to another nucleic acid when these sequences are arranged in a functional relationship. This can be in the form of a gene (or coding sequence) and one or more control sequences linked such that the gene is expressed when an appropriate target gene is combined with a control sequence(s). For example, when DNA encoding a pre-sequence or secretion leader sequence is expressed as a pre-protein participating in the secretion of a polypeptide, it is linked operably to DNA encoding the polypeptide. If a promoter or an enhancer affects (stimulates) the transcription of a coding sequence, it is linked operably to that a coding sequence. If a coding sequence encoding a ribosome binding region affects the transcription of a protein-coding sequence, it is operably linked to that coding sequence. If a sequence encoding a ribosome binding region is arranged to facilitate translation of a coding sequence, it is operably linked to that coding sequence. Generally “operably linked” means that linked DNA sequences are contiguous and, in case of secretion leader, it is contiguous and is in the same reading frame (as the sequence encoding the mature protein). However, an enhancer does not need to be immediately adjacent to the coding sequence. The physical linkage of these sequences is achieved by ligation at a convenient restriction enzyme site. If a restriction enzyme site does not exist, a synthetic oligonucleotide adaptor or linker is used according to conventional methods to create a site.

The inventive recombinant vector in which the shuttle vector and a target gene are operably linked, can be transformed into an appropriate host cell by conventional methods. A preferred host cell is a bacterium of the genus Rhodococcus. In the present invention, the useful Rhodococcus bacteria species is not limited in any specific way, but, can generally be selected from the group consisting of Rhodococcus coprophilus, R. equi, R. erythropolis, R. fascians, R. globerula, R. rhodnii, R. rhodochrous, R. ruber and R. rubrum.

### EXAMPLES

Hereinafter, the present invention will be described in more detail by examples. It is to be understood, however, that these examples are given to more fully describe the present invention and are not to be construed as limitations.

### Example 1

### Isolation of Cryptic Plasmid Derived from Rhodococcus erythropolis IAM1484

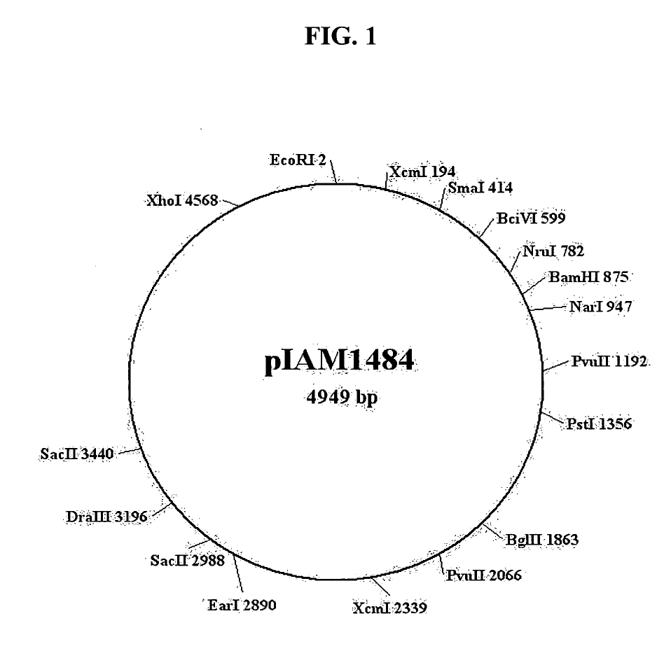

In this example, the cryptic plasmid was isolated from Rhodococcus erythropolis IAM1484 (Rhodococcus erythropolis IAM1484; ACTC 15961). First, Rhodococcus erythropolis IAM1484 strain was cultured in 5 ml YEPD medium (bactopeptone 2%, yeast extracts 1%, glucose 2%) in test tubes at 30° C. overnight. The cultures were centrifuged at 200 rpm to yield a cell pellet. The cell pellet was washed twice with 500 μl of TE buffer (Tris 10 mM, EDTA 1 mM, pH 8) and again pelleted by centrifugation. This pellet was resuspended with 500 μl TE buffer, and 1 mg of lysozyme was added and the mixture mixed adequately. The suspension was incubated at 37° C. for 1 hr to weaken the cell walls, followed by isolating and purifying the plasmid (pIAM1484; FIG. 1) by a conventional process (Sambrook, J. et al., Molecular Cloning: A Laboratory Manual, 2nd Edition, Cold Spring Harbor Press, Cold Spring Harbor, N.Y., 1989).

### Example 2

### Cloning of Cryptic Plasmid

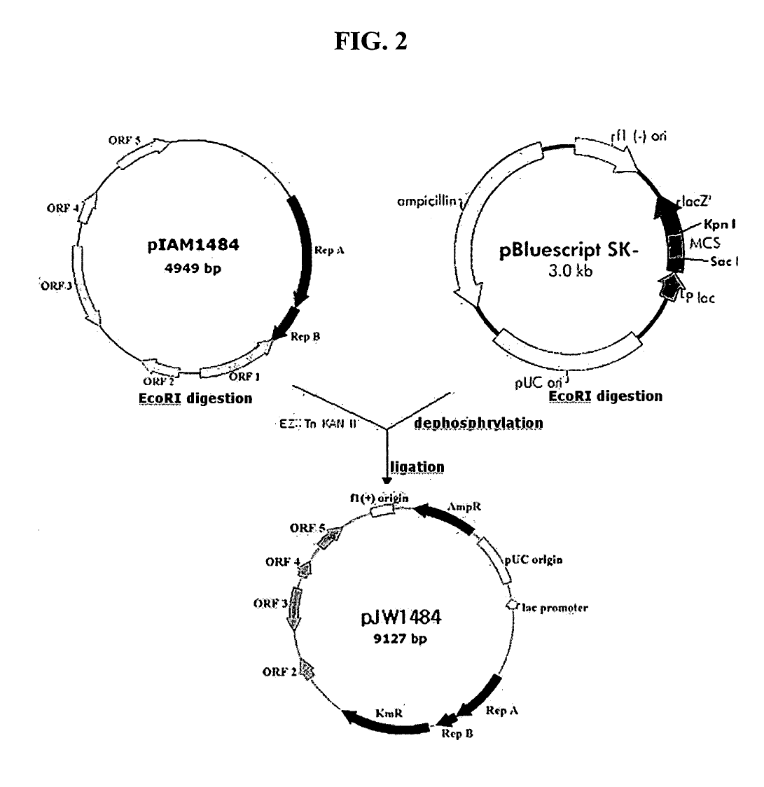

To simplify the handling of the cryptic plasmid, pIAM1484 isolated in Example 1 was combined with an origin of replication (ori) in E. coli and pBluescript SK(−) having an ampicillin resistance gene (FIG. 2). The precise location of the origin of replication of the cryptic plasmid was not known. However, one EcoRI restriction enzyme site existed in the plasmid of the present invention, so that one linear plasmid could be obtained by digesting the cryptic plasmid with the EcoRI restriction enzyme. The plasmid digested with EcoRI was ligated with pBluescript SK(−) plasmid which had also been digested with EcoRI and was dephosphorylated for introduction into E. coli, thus obtaining a plasmid of about 8 kb.

### Example 3

### Preparation of pJW1484

The entire sequence of the cryptic plasmid was determined using EZ::TN™ [KAN-2] Insertion Kits (Epicentri Co.) as disclosed in Goryshin, I Y and Reznikoff, W S, J. Biol. Chem. 273:7367, 1998. The colonies in which a transposon was added at 500 bp intervals in the cryptic plasmid region were selected and transformed, respectively, into Rhodococcus erythropolis LG12. The success vs. failure of transformation was the basis for determining presence of an important region for replication.

Colonies which could not be transformed arose from transformation with plasmids in which transposon was inserted at 300 bp, 800 bp, and 1300 bp, respectively from the start region of cryptic plasmid. By Blast searching, the sequence from about nucleotide 706 to about nucleotide 1877 of the sequence running from nucleotide 1 to about nucleotide 2000 was assumed to encode a binding protein region and replicase responsible for replicating the plasmid directly, and the region from nucleotide 1 to about nucleotide 706 region was identified as an origin or replication. The plasmid in which transposon comprising a kanamycin resistance gene is located 2 kb from an EcoRI site of the cryptic plasmid was isolated, and named pJW1484 (FIG. 2).

One μg of isolated pJW1484 DNA was introduced into Rhodococcus erythropolis LG12 (KCTC 10838BP) by electroporation using a cuvette having a 1 mm gap at 1250 Volt, 25 μF (Gene Pulser II System from Biorad).

### Example 4

### Preparation of pLG1484

To minimize the shuttle vector size, sequences unrelated to replication in E. coli and Rhodococcus were deleted from the pJW1484 plasmid prepared in Example 3, and the region assumed to be related to replication and antibiotic resistance in Rhodococcus was selected. In other words, PCR was performed using pJW1484 as a template with primers having the sequences, SEQ ID NO:2 and SEQ ID NO:3 (below) to obtain about 3.3 kb of DNA PCR product. The 5′ end of the primer was modified with phosphate.

| | |
|---|---|
| Cryptic forward (forward primer): | |
| 5′-P-GACACATTTCGACCGAAGGACATC-3′ | (SEQ ID NO:2) |
| Kan reverse (reverse primer): | |
| 5′-P-CACGGTTGATGAGAGCTTTGTTGTAG-3′ | (SEQ ID NO:3) |

Also, PCR was performed using pJW1484 as a template with primers having the sequences, SEQ ID No:4 and SEQ ID NO:5 (below) to obtain the region related to replication and antibiotic resistance in E. coli. As a result, a 2.8 kb DNA fragment was obtained.

| | |
|---|---|
| M13 forward RC (forward primer): | |
| 5′-ACTGGCCGTCGTTTTAC-3′ | (SEQ ID NO:4) |
| M13 reverse RC (reverse primer): | |
| 5′-CATGGTCATAGCTGTTTCC-3′ | (SEQ ID NO:5) |

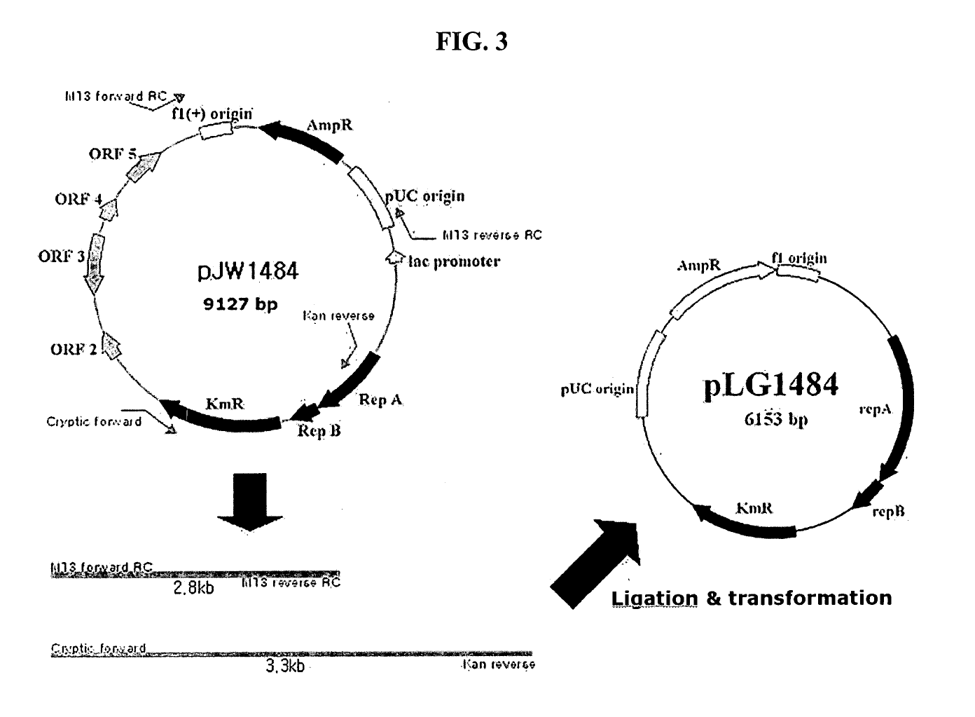

Each PCR product was blunt-ended using T4 DNA polymerase, ligated using T4 DNA ligase, and introduced to E. coli. The cloned 6153 bp plasmid named pLG1484 (FIG. 3) was deposited in the International Depository, KCTC on Jan. 21, 2005 and assigned the accession number KCTC 10770BP. The DNA sequence of pLG1484 is SEQ ID NO: 1.

### Example 5

### Replication Stability of pLG1484

Rhodococcus erythropolis LG12 cells comprising pLG1484 prepared as in Example 4 were cultured in liquid YEPD medium containing 40 μg/mL of kanamycin until late exponential phase. Cells at a 1:100 dilution were inoculated into 50 mL of liquid YEPD medium without antibiotics and cultured at 30° C. 24 hours later, the cells were again diluted 100-times and inoculated in 50 mL of liquid YEPD medium without antibiotics. As the procedure was repeated, cells obtained at each step were plated on solid YEPD medium without antibiotics, and generation time was measured by colony counting.

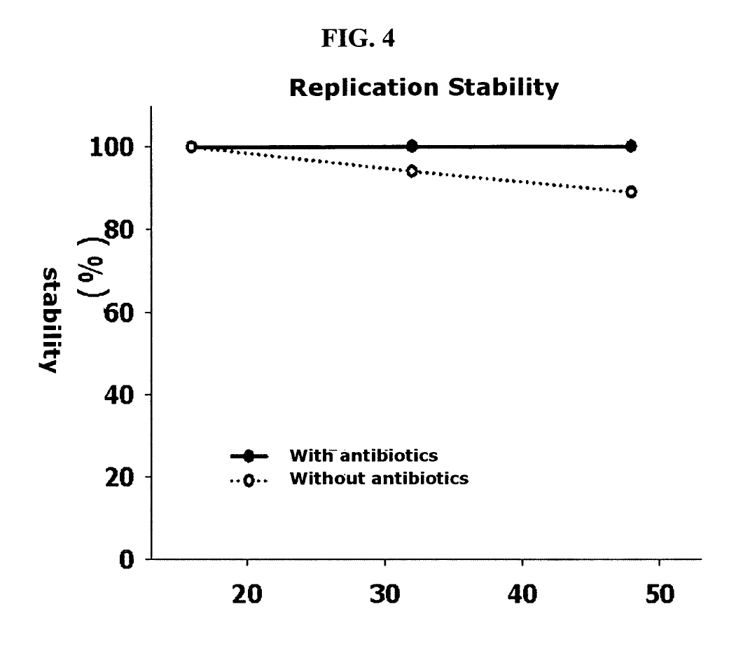

Afterwards, 100 colonies produced on the solid medium were inoculated into solid YEPD medium without antibiotics and cultured for 24 hours. Each colony was inoculated in solid YEPD medium containing 40 μg/mL of kanamycin. Then, the replication stability of pLG1484 was examined by measuring the number of kanamycin-sensitive colonies among the 100 colonies (FIG. 4).

The result are presented in FIG. 4. Rhodococcus erythropolis LG12 cells comprising pLG1484 according to the present invention showed excellent replication stability without selection by antibiotics. About 0.22% of whole cells per one replication in medium excluding antibiotics were cells without the shuttle vector pLG1484. As compared to the report that pMVS301 plasmid in Rhodococcus sp. AS-50-1 (Vogt Singer & Finnerty, J. Bacteriol. 170:638, 1998) and pK4 plasmid in Rhodococcus rhodochrous ATCC 12674 (Komeda, H. et al., Proc. Natl. Acad. Sci. USA 93:10572, 1996) were deactivated at the rate of 1˜1.5%, it was concluded that pLG1484 according to the present invention had high replication stability.

### Example 6

### Host Range of pLG1484

Rhodococcus globerulla (KCCM 40036), Rhodococcus rhodochrous (KCCM 40120), Rhodococcus equi (KCCM 12541) and Rhodococcus ruber (KCCM 41053) obtained from KCCM (Korea Culture Center of Microorganism) were transformed with pLG1484, respectively, to identify the host range of Rhodococcus-E. coli shuttle vector pLG1484. Cells of each species were cultured, mixed with 1 μg of recombinant plasmid pLG1484, and transformation was carried out using a cuvette for electroporation, having 1 mm gap under conditions of 1250 Volt, 25 μF. Transformants were determined by acquisition of resistance to 50 μg/mL of kanamycin.

TABLE 1

| | Transformants/ |
|---|---|
| Host cells | 1 μg pLG1484 DNA |
| R. erythropolis LG12 | 4.3 × 10⁴ |
| R. equi KCCM 12541 | 3.2 × 10⁵ |
| R. globerulla KCCM 40036 | 2.6 × 10⁴ |
| R. rhodochrous KCCM 40120 | 0 |
| R. ruber KCCM 41053 | 0 |

As a result, as indicated in Table 1, pLG1484 has a broad host range because it replicated in at least three species, Rhodococcus equi, Rhodococcus globerulla, and Rhodococcus erythropolis, which have a rather distant evolutionary relationship. Transformation efficiency of Rhodococcus equi was 7 times greater than that of Rhodococcus erythropolis, and that of Rhodococcus globerulla was half of that of Rhodococcus erythropolis. No transformation of Rhodococcus rhodochrous and Rhodococcus ruber was observed. From these results, it was concluded that the shuttle vector pLG1484 according to the present invention had a broader host range than did other known shuttle vectors for Rhodococcus:

(1) pAN12 (PCT Pub. WO02/055709A2),

(2) pDA71 (Dabbs E R et al., Plasmid 23:42, 1990), and

(3) pFAJ2600 (Demot et al., Microbiol. 146:3137, 1997).

As described more in detail, the present invention provides (1) a shuttle vector that can replicate bidirectionally in E. coli and Rhodococcus, (2) a recombinant vector in which a target gene is operably linked to the shuttle vector, and (3) a microorganism transformed with the recombinant vector. The shuttle vector according to the present invention has many advantages for use in methods of gene manipulation due to its relatively small size and the presence of strong replication proteins. Also, it has the requisite stability for fermentation processes or biotransformation processes because it can be replicated effectively in medium lacking in antibiotics due to its high replication stability in Rhodococcus. In addition, the shuttle vector has a broad host range for many species of the genus Rhodococcus, making it useful for the cloning of target genes derived from the genus Rhodococcus and various cells.

While the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

What is claimed is:

1. A Rhodococcus-E. coli shuttle vector comprising a DNA origin of replication derived from Rhodococcus erythropolis, a DNA origin of replication derived from E. coli, an ampicillin resistance gene selection marker expressed in E. coli, and a kanamycin resistance gene selection marker expressed in bacteria of the genus Rhodococcus.

2. The Rhodococcus-E. coli shuttle vector according to claim 1, wherein the DNA origin of replication derived from Rhodococcus erythropolis is derived from a plasmid of Rhodococcus erythropolis IAM1484.

3. The Rhodococcus-E. coli shuttle vector according to claim 2, wherein the plasmid is pIAM1484.

4. The Rhodococcus-E. coli shuttle vector according to claim 1, wherein the shuttle vector is pJW1484.

5. The Rhodococcus-E. coli shuttle vector according to claim 1, wherein the nucleotide sequence of the shuttle vector has at least 95% sequence homology with SEQ ID NO:1.

6. The Rhodococcus-E. coli shuttle vector according to claim 5, wherein the shuttle vector is pLG1484.

7. A recombinant vector comprising a target gene operably linked to the pJW1484 shuttle vector of claim 4.

8. A microorganism transformed with the recombinant vector of claim 7.

9. The transformed microorganism according to claim 8 which is a bacterium of the genus Rhodococcus.

10. The transformed microorganism according to claim 9, wherein the Rhodococcus bacterium is a member of a species selected from the group consisting of R. coprophilus, R. equi, R. erythropolis, R. fascians, R. globerula, R. rhodnii, R. rhodochrous, R. ruber and R. rubrum.

11. A recombinant vector comprising a target gene operably linked to the pLG 1484 shuttle vector of claim 6.

12. A microorganism transformed with the recombinant vector of claim 11.

13. The transformed microorganism according to claim 12 which is a bacterium of the genus Rhodococcus.

14. The transformed microorganism according to claim 13, wherein the Rhodococcus bacterium is a member of a species selected from the group consisting of R. coprophilus, R. equi, R. erythropolis, R. fascians, R. globerula, R. rhodnii, R. rhodochrous, R. ruber and R. rubrum.
